(19) United States
(12) Patent Application Publication
(10) Pub. No.: US 2003/0017502 A1
(43) Pub. Date: Jan. 23, 2003

(54) Methods involving the use of capG as a diagnostic marker and methods for the identification of capG modulators for the treatment and prevention of osteoporosis and related disease states

(76) Inventor: C. Patrick McAtee, Pennington, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/098,931

(22) Filed: Mar. 15, 2002

Related U.S. Application Data
(60) Provisional application No. 60/276,250, filed on Mar. 15, 2001.

(51) Int. Cl. G01N 33/53; G01N 33/567

(52) U.S. Cl. 435/7.1; 435/7.21

(57) ABSTRACT

The present invention relates to the identification of capG as a critical enzyme in the process of RANKL-induced osteoclast-activation. The present invention includes modulators of capG and assays for the identification of such modulators, as well as the use of such modulators in the treatment and prevention of osteoporosis and related disease states.

This application claims the benefit of provisional application U.S. Serial No. 60/276,250, filed Mar. 15, 2001.

### FIELD OF THE INVENTION

The present invention relates to methods involving the use of capG as a diagnostic marker and methods for the identification of capG modulators. The capG modulators are useful for the treatment and prevention of osteoporosis and related disease states.

### BACKGROUND OF RELATED TECHNOLOGY

The osteoclast is a terminally differentiated cell derived from monocytic/macrophage lineage which resorbs bone as part of the normal process of skeletal modeling and remodeling. In contrast to precursor cells, only fully differentiated mature osteoclasts are able to resorb bone. Increased osteoclastic bone resorption has been linked to the pathogenesis of several skeletal disorders, most notably post-menopausal osteoporosis. As activated osteoclasts move over the bone surface to initiate new sites of bone resorption, cytoskeletal rearrangements lead to the formation of unique cell adhesion structures called podosomes which attach to the bone matrix via intermediate steps. Podosomes consist of an F-actin core surrounded by the actin-binding proteins vinculin, talin, and α-actinin (Marchisio, P. C. et al., “Cell-substratum interactions of cultured avian osteoclasts is mediated by specific adhesion structures”. J. Cell Biol., 99:1696-1705 (1984)). Osteoclasts attached to bone form a tight sealing zone resulting in an enclosed resorption lacunae. Localized membrane ruffling occurs at the cellular surface facing the bone, allowing the transport of protons and degradative enzymes within the resorption lacunae. One compound which is known to be involved with such ruffled border formation is gelsolin, although its precise function is not known.

Gelsolin is a member of the large and diverse gelsolin superfamily of proteins. Some gelsolin family proteins both sever and cap actin filaments, while others only cap. Some can also bundle filaments. Gelsolin itself is an actin-binding protein which controls the length of actin filaments in vitro, and cell shape and motility in vivo by a variety of mechanisms. (Cunningham, C. C. et al., “Enhanced motility in NIH3T3 fibroblasts that overexpress gelsolin”. Science, 251:1233-1236 (1991)). Gelsolin severs assembled actin filaments, caps the fast-growing plus ends, and promotes growth of actin filament by creating nucleation sites (Stossel, T. P. et al., “From signal to pseudopod. How cells control cytoplasmic actin assembly”. J. Biol. Chem., 264:18261-18264 (1989)). Severing and capping are regulated by calcium ions and pH, which synergistically activate gelsolin's binding to actin. Binding of gelsolin to phosphinositides causes the release of gelsolin from the filament end (uncapping), providing a site for rapid monomer addition (Jamney, P.A. and T. P. Stossel, “Modulation of gelsolin function by polyphosphoinositol(4,5)-bisphosphate”. Nature, 325:362-364 (1987); Janmey, P. A. et al., “Polyphosphoinositide micelles and polyphosphoinositide-containing vesicles dissociate endogenous gelsolin-actin complex and promote actin assembly from the fast growing end of actin filaments blocked by gelsolin”. J. Biol. Chem., 262:12228-12236 (1987)).

One member of the gelsolin superfamily of proteins, capG (previously known as macrophage capping protein, gCap39, and Mbh1) reflects sequence similarity to gelsolin (Onoda, K. and H. L. Yin, “gCap39 is phosphorylated. Stimulation by okadaic acid and preferential association with nuclei”. J. Biol. Chem., 268:4106-4112 (1993); Prendergast, G. C. and E. B. Ziff, “Mbh 1: A novel gelsolin/severin-related protein which binds actin in vitro and exhibits nuclear localization in vivo”. EMBO, 10:757-766 (1991)) and has been shown to play an important role in macrophage function. CapG caps barbed ends and nucleates actin assembly, but does not sever, which is different from gelsolin. Like gelsolin, capG requires micromolar calcium and is inhibited by phosphoinositides, but unlike gelsolin, capG dissociates from the ends of actin filaments at low calcium concentrations. (Young, C. L. et al., “Calcium regulation of actin filament capping and monomer binding by macrophage capping protein”. J. Biol. Chem., 269:13997-14002 (1994)).

The amino acid sequence of capG is composed of three to six repeats found in gelsolin and is similar to the protozoan proteins severin and fragmin. (Dabiri, G. A. et al., “Molecular cloning of human macrophage capping protein cDNA. A unique member of the gelsolin/villin family expressed primarily in macrophages”. J. Biol. Chem., 267:16545-16552 (1992)). CapG expression represents 1% of the total cytoplasmic protein in macrophages (“Comparisons of CapG and gelsolin-null macrophages: demonstration of a unique role for CapG in receptor-mediated ruffling, phagocytosis, and vesicle rocketing.” J. Cell Biol., 154:775-784 (2001)). CapG serves important functions in actin based macrophage motility that are distinct from those of gelsolin. CapG is required for receptor-mediated membrane ruffling.

Furthermore, it is known that osteoclast precursor cells possess a receptor, receptor activator of NF-κB (RANK), that recognizes a ligand (RANKL) which leads to differentiation (Suda, T. et al., “Modulation of Osteoclast Differentiation and Function by the New Members of the Tumor Necrosis Factor Receptor and Ligand Families”. Endocr. Rev., 20:345-357 (1999); Takahashi, N. et al., “A New Member of Tumor Necrosis Factor Ligand Family, ODF/OPGL/TRANCE/RANKL, Regulates Osteoclast Differentiation and Function”. Biochem. and Biophys. Res. Comm., 256:449-455 (1999)). The RANKL receptor is a member of the tumor necrosis factor (TNF) family and is a specific inducer of osteoclastogenesis. (Simonet W. S., et al., “Osteoprotegerin: a novel secreted protein involved in the regulation of bone density”. Cell, 89(2):309-319 (1997); Kong, Y. Y. et. al., “OPGL is a key regulator of osteoclastogenesis, lymphocyte development and lymph-node organogenesis”. Nature, 397(6717):315-323 (1999)). Additionally, RANKL and RANK knockout mice have shown osteopetrosis, indicating their importance in osteoclast differentiation.

Although PI3kinase, rhoA, gelsolin, and pp60c-src have been shown to be essential for cytoskeletal rearrangement and osteoclast-mediated bone resorption, little is known of the signal transduction events initiated through the RANKL receptor. (Nakamura, I. et al., “Phosphatidylinositol-3 kinase is involved in ruffled border formation in osteoclasts”. J Cell Physiol., 172(2):230-239 (1997); Chellaiah, M. A. et al., “Rho-A is critical for osteoclast podosome organization, motility, and bone resorption”. J Biol. Chem., 275(16):11993-20002 (2000); Schwartzberg, P. L. et al., “Rescue of osteoclast function by transgenic expression of kinase-deficient Src in src−/− mutant mice”. Genes Dev., 11(21):2835-44 (1997)).

Accordingly, there exists a continuing need to identify compounds involved in the RANKL pathway, as well as modulators therefore, which are useful for the identification, prevention and treatment of osteoporosis and related disease states. The present invention is directed towards meeting these and other needs.

### SUMMARY OF THE INVENTION

It has now been found that capG is a critical enzyme in the process of RANKL-induced osteoclast-activation. Accordingly, the present invention is directed to the use of capG as a protein marker for diagnostic purposes and as a molecular target for drug discovery. The present invention is further directed to modulators of capG in the RANKL pathway, methods for the identification of such modulators and methods of using such modulators in pharmaceutical formulations in the prevention and treatment of osteoporosis and related disease states.

In one aspect, the present invention is directed to an assay for identifying a compound that modulates the activity of capG, including the steps of: (1) providing a cell expressing capG; (2) contacting the cell expressing capG with a test compound; and (3) determining whether the test compound interacts with capG. The assay may be a cell-based assay or a cell-free assay, such as a ligand-binding assay. The test compound may modulate the activity of capG, and may be a capG antagonist or a capG agonist. Further, the test compound may bind to capG, and may inhibit the activity of capG. The assay may be for identifying compounds which will be useful for the treatment of osteoporosis.

In another aspect, the present invention is directed to a method for the treatment of osteoporosis, which includes administering to a patient in need thereof a therapeutically effective amount of a compound which is identified by this assay.

In another aspect, the present invention is directed to a method for the treatment of osteoporosis, which includes the steps of: (1) identifying a patient suffering from osteoporosis; and (2) administering to the patient a therapeutically effective amount of a modulator of capG. The patient may be identified as suffering from osteoporosis by measuring the expression level of capG in the patient.

In another aspect, the present invention is directed to a method for the prevention of osteoporosis, which includes the steps of: (1) identifying a patient at risk for osteoporosis; and (2) administering to the patient a therapeutically effective amount of a modulator of capG. The patient may be identified as being at risk for osteoporosis by measuring the expression level of capG in the patient.

In another aspect, the present invention is directed to a method for the prevention of osteoporosis, including the steps of: (1) identifying a patient at risk for osteoporosis; and (2) determining if the patient has increased expression of capG, wherein the increased expression is measured relative to the expression of capG in a patient not at risk for osteoporosis.

In another aspect, the present invention is directed to a method of decreasing the differentiation of osteoclast precursor cells into osteoclast cells, which includes the step of contacting the osteoclast precursor cells with a capG modulator.

In another aspect, the present invention is directed to a compound capable of modulating the activity of capG. This compound may be identified by the steps of: (1) providing a cell expressing capG; (2) contacting the cell expressing capG with the compound; and (3) determining whether the compound modulates the activity of capG. The compound may be a capG antagonist, agonist, and may bind to capG.

### BRIEF DESCRIPTION OF THE DRAWINGS

FIG. 1 shows a two-dimensional gel of RANKL-stimulated RAW264 cells stained with Sypro Ruby Red.

FIG. 2a shows a two-dimensional anti-phosphotyrosine Western blot of RAW264 cells grown in the absence of RANKL.

FIG. 2b shows a two-dimensional anti-phosphotyrosine Western blot of RAW264 cells stimulated with RANKL.

FIG. 2c shows a two-dimensional anti-phosphotyrosine Western blot of RAW264 cells grown in the absence of RANKL and pretreated with PP1.

FIG. 2d shows a two-dimensional anti-phosphotyrosine Western blot of RAW264 cells grown stimulated with RANKL following pretreatment with PP1.

FIG. 3a shows a two-dimensional anti-phosphotyrosine Western blot of RAW264 cells grown in the absence of RANKL.

FIG. 3b shows a two-dimensional anti-phosphotyrosine Western blot of RAW264 cells stimulated with RANKL.

FIG. 3c shows a two-dimensional anti-phosphotyrosine Western blot of RAW264 cells grown in the absence of RANKL and pretreated with wortmannin.

FIG. 3d shows a two-dimensional anti-phosphotyrosine Western blot of RAW264 cells stimulated with RANKL following pretreatment with wortmannin.

FIG. 4 shows a DNA alignment of capG to gelsolin.

FIG. 5 shows a three-dimensional alignment of capG with actin.

FIG. 6 shows comparative contact binding areas of gelsolin versus capG with actin.

FIG. 7 shows a confocal microscopy image of mouse osteoclasts on ivory which have been differentiated with RANK-L and M-CSF. The cells have been stained for actin and gelsolin.

FIG. 8 shows a confocal microscopy image of mouse osteoclasts on ivory which have been differentiated with RANK-L and M-CSF. The cells have been stained for actin and capG.

FIG. 9 shows the bone mineral density analysis of capG (−/−) mice versus wild type mice.

### DETAILED DESCRIPTION OF THE INVENTION

It has now been found that capG is a critical enzyme in the process of RANKL-induced osteoclast-activation. Accordingly, the present invention is directed to the use of capG as a protein marker for diagnostic purposes and as a molecular target for drug discovery. The present invention is further directed to modulators of capG in the RANKL pathway, methods for the identification of such modulators and methods of using such modulators in pharmaceutical formulations in the prevention and treatment of osteoporosis and related disease states.

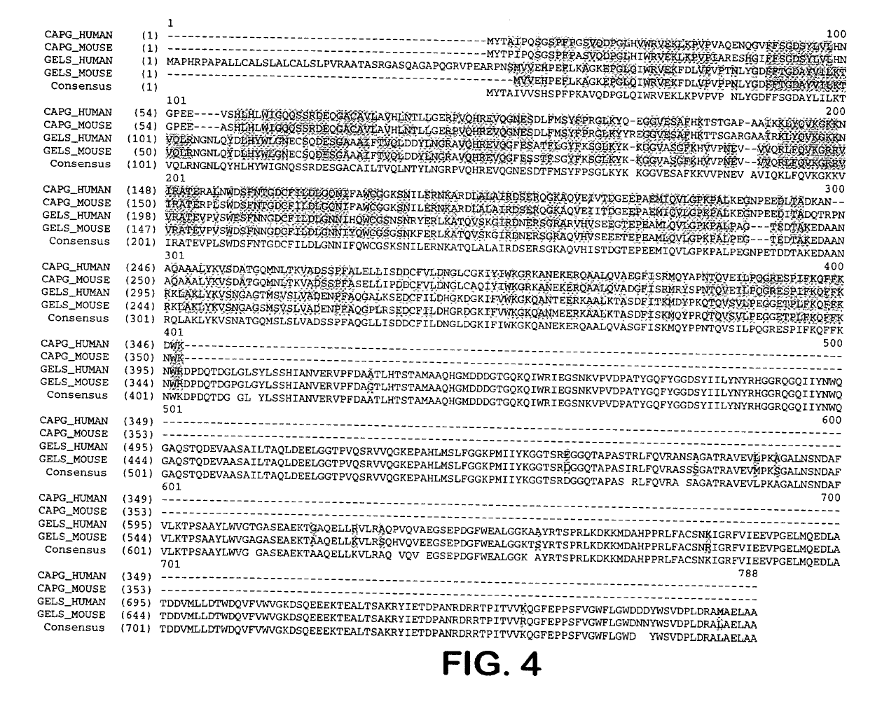

CapG (also known in the art as macrophage capping protein, gCap39, and Mbh1), caps but does not sever barbed ends of actin filaments and nucleates actin assembly. CapG requires micromolar calcium, is inhibited by phosphoinositides, and dissociates from the ends of actin filaments at low calcium concentrations. Further, it reflects sequence similarity to gelsolin, as shown in FIG. 4. By way of example only, human capG is a protein having the amino acid sequence shown in FIG. 4 (Dabiri, G. A. et al., “Molecular cloning of human macrophage capping protein cDNA. A unique member of the gelsolin/villin family expressed primarily in macrophages”. J. Biol. Chem., 267(23): 16545-16552 (1992), encoded by the nucleic acid sequence set forth in Mishra, V. S. et al., “The human actin-regulatory protein capG: gene structure and chromosome location”. Genomics, 23(3):560-565. Additionally, mouse capG may be used in the present invention, the amino acid sequence for which is shown in FIG. 4, and which is encoded by the nucleic acid sequence set forth in Prendergast, G. C. and E. B. Ziff, “Mbh 1: A novel gelsolin/severin-related protein which binds actin in vitro and exhibits nuclear localization in vivo”. EMBO, 10(4):757-766 (1991)). However, the present invention is not limited to the use of human or murine capG, and may include the use of capG from any suitable organism.

Osteoclast precursor cells possess a receptor, receptor activator of NF-κB (RANK), that recognizes a ligand (RANKL) which leads to differentiation. RANK is a member of the tumor necrosis factor (TNF) family and is a specific inducer of osteoclastogenesis. Additionally, RANKL and RANK knockout mice have shown osteopetrosis, indicating their importance in osteoclast differentiation.

It will be apparent to one of skill in the art that conventional screening assays may be used in methods of the present invention for the identification of capG modulators. Such screening assays include, but are not limited to, phalloidin binding fluorescence assays, actin binding, actin bundling sedimentation assays, and confocal microscopy assays utilizing osteoclast substrates such as ivory.

As used herein, the term “modulators” is given the conventional meaning of the term, and includes, but is not limited to, agonists, antagonists, and compounds the bind directly and indirectly to capG and the calcium binding sites within the capG protein.

CapG modulators of the present invention are suitable for use in the prevention and treatment of osteoporosis and related disease states. A compound which acts as such a modulator may be administered for therapeutic use as a raw chemical or may be the active ingredient in a pharmaceutical formulation.

When provided in pharmaceutical formulations, a compound acting as a capG modulator, or a physiologically acceptable salt or solvate thereof, may be provided together with one or more pharmaceutically acceptable carriers or excipients. The carrier(s) or excipient(s) must be acceptable in the sense of being compatible with the other ingredients of the formulation and not deleterious to the recipient thereof.

Pharmaceutical formulations of the present invention may be formulated for administration by any suitable route, including, but not limited to oral, rectal, nasal, topical (including buccal and sublingual), vaginal or parental (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

When desired, the above described formulations adapted to give sustained release of the active ingredient may be employed. The compounds and pharmaceutical compositions of the invention may also be used in combination with other therapeutic agents.

Proteomic analysis of RANKL-induced signal transduction intermediates from RAW 264 cells (murine macrophage cell line) was conducted as set forth in the Example below. From this analysis, it can be seen that RANKL induces specific tyrosine phosphorylation of capG, establishing the importance of capG in the process of RANKL-induced osteoclast activation. As such, capG is an important target in the treatment and prevention of osteoporosis.

### EXAMPLE

### Identification of capG in the RANKL Pathway

RAW 264 cells were obtained from Bristol Myers Squibb Department of Metabolic Diseases. The cells were grown in minimal essential media supplemented with 5% fetal bovine serum and 1% nonessential amino acids. For assay purposes, RAW 264 cells were starved for 5 hours in serum free media and then cultured in media containing 2% fetal bovine serum and RANK ligand. When inhibitors were used, as set forth below, the cells were pre-exposed to the inhibitor for one hour prior to RANKL stimulation.

To identify RANKL specific tyrosine phosphorylation, RAW264 cells were treated with RANKL in the presence and absence of PP1 [4-Amino-5-(4-methylphenyl)-7-(t-butyl)pyrazolo[3,4-d]pyrimidine], a specific inhibitor of pp60src and wortmannin, a specific inhibitor of PI3kinase (Lakkakorpi, P. T. et al., “Phosphatidylinositol 3-kinase association with the osteoclast cytoskeleton, and its involvement in osteoclast attachment and spreading”. Exp. Cell Res. 237(2):296-306 (1997)), and cell lysates evaluated by proteomic analysis. Cell lysates were separated by two-dimensional electrophoresis in duplicate and transferred to PVDF membranes.

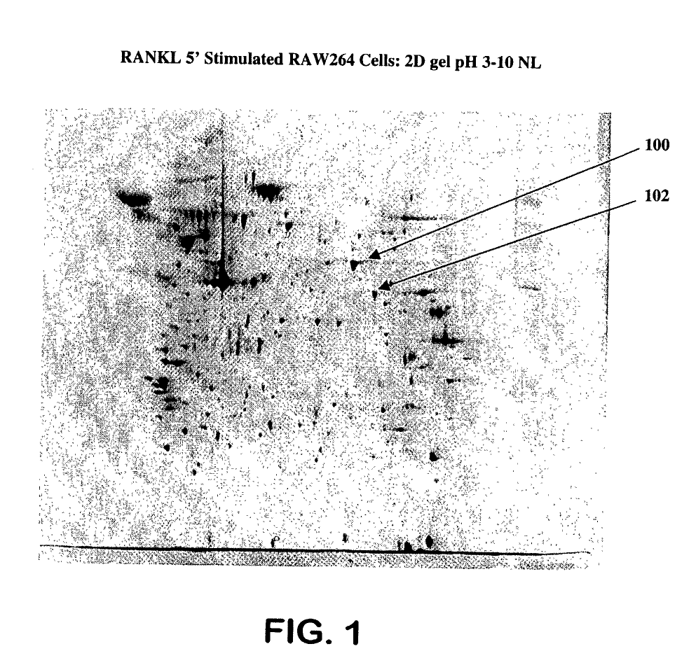

One PVDF membrane was stained with Sypro Ruby Red, as set forth in Valdes, I. et al. “Novel procedure for the identification of proteins by mass fingerprinting combining two-dimensional electrophoresis with fluorescent SYPRO red staining”. J. Mass. Spectrom. 35(6):672-682 (2000), for subsequent proteomic analysis with the corresponding membrane analyzed by western blotting using anti-phosphotyrosine antibody. A Sypro Ruby Red stained 2-D of RANKL stimulated RAW 264 cell lysates is shown in FIG. 1. CapG 102 is identified in FIG. 1 and α-enolase 100 is identified as a landmark.

Isoelectric focusing was carried out in Pharmacia IPG strips, pH 3-10 nonlinear gradient for approximately 150,000 Vhr. Following equilibration for 15 min in 10% glycerol, 50 mM DTT, 2.3% SDS, and 62.5 mM Tris (pH 6.5), the IPG strip was layered onto the top of a 10% acrylamide slab gel (1.00 mm thick), and SDS slab gel electrophoresis was carried out for 5 hrs. at 20 watts/gel. The slab gels were transferred overnight to PVDF membrane which were then were fixed in a solution of 10% acetic acid-40% methanol for 30 min. followed by staining overnight with the fluorescent dye Sypro Ruby Red (Molecular Probes, Eugene, Oreg.) as described in the manufacturers protocol. Maximal fluorescence incorporation occurred within 4 hours.

For Western blots, the PVDF membranes were blocked for greater than 2 hours with 1% bovine serum albumin (BSA) (w/v) in 1% Tween-Tris buffered saline (TTBS) (v/v), rinsed in TTBS, incubated with primary antibody diluted 1:2,500 in 1% BSA-TTBS for 2 hours, rinsed in TTBS, and incubated with secondary antibody diluted 1:5,000 in TTBS for 1 hour. The blot was rinsed with TTBS, and treated with ECL (Pharmacia-Amersham Biotech, Piscataway, N.J.). Images were generated using a BioRad Fluor-S Max imaging system. The images were then interpreted using PDQuest 6.1 software (BioRad Laboratories Hercules, Calif.). Samples were selected for in-gel digestion based upon information obtained from digital images generated from chemilumenescent stained western blots compared to Sypro fluorescent stained gel images.

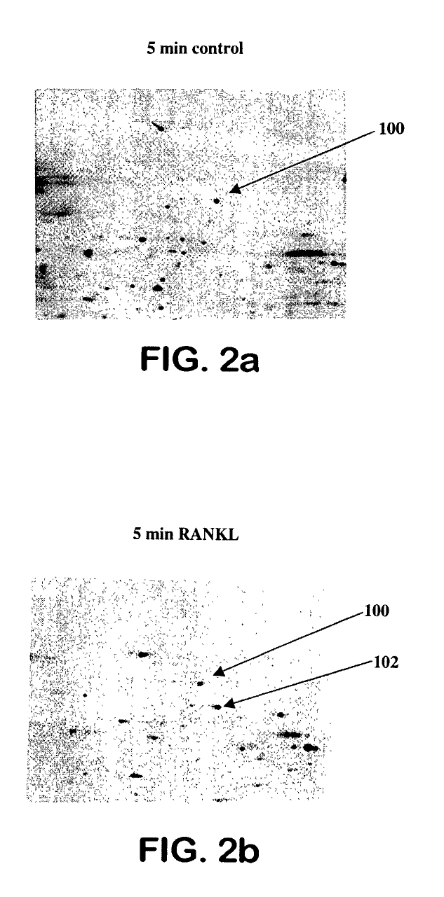

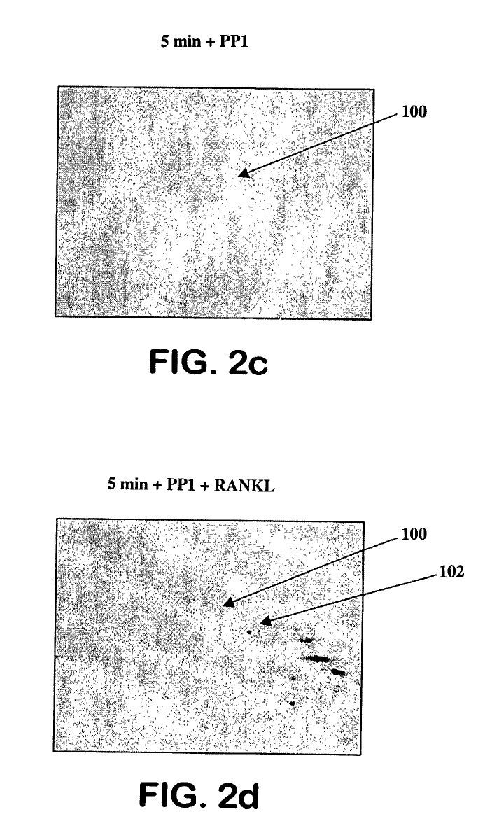

Western blot data showing phosphotyrosine activity in the presence or absence of PP1 is shown in FIGS. 2a-2d. FIG. 2a shows an unstimulated (control) blot with α-enolase 100 identified as a landmark. Stimulation with RANKL causes capG 102 phosphorylation (FIG. 2b). As seen in FIGS. 2c and 2d, PP1 has a pronounced effect on the phosphorylation pattern on the control unstimulated cells, as well as on the RANKL stimulated RAW 264 cells, respectively. The increase in capG phosphorylation in RANKL-stimulated cells and the decrease in capG phosphorylation in RANKL-stimulated cells the presence of PP1 indicates that capG is a critical enzyme in the RANKL pathway.

Identities of protein spots selected from corresponding Sypro Red stained 2-D gels shown in FIGS. 1-3 were digested in situ with trypsin and then analyzed by ESI-MS. Selected protein spots were excised and washed twice with water for 15 min. Samples were then dried under vacuum in a Savant SpeedVac. The samples were then reduced and alkylated and the gel pieces were washed with 50% acetonitrile: 100 mM ammonium bicarbonate (v/v) and dried again under vacuum. The gel pieces were then rehydrated with ammonium bicarbonate containing 12.5 ng trypsin and incubated overnight at 37° C. Following digestion, the gel pieces were extracted with 50% acetonitrile: 100 mM ammonium bicarbonate (v/v) and the supernatants dried under vacuum. The dried material was resuspended in formic acid for mass spectral analysis.

Following the extraction of peptides from the gel pieces, the samples were evaporated to dryness (Model AES2010, Savant Instruments, Holbrook, N.Y.). The peptides were dissolved in 5% formic acid, vortexed, sonicated, and then briefly centrifuged to settle insoluble matter. The samples were then loaded onto capillaries packed with a stirred slurry of POROS R2/H (PE-Biosystems, Framingham, Mass.) using an argon pressure reservoir as previously described.

The capillaries were pre-equilibrated with >10 column volumes of mobile phase A (A=0.2% isopropanol, 0.1% acetic acid, 0.001% trifluoroacetic acid) prior to the sample loading process. The chromatographic separation was preformed with a gradient of increasing organic concentration of 0% B-100% B (B=A+80% acetonitrile) in 45 min at an initial applied pressure of 22 bar generated using a binary HPLC pump (Model 1100, Hewlett-Packard, Palo Alto, Calif.) flowing at 250 μl per min. prior to the split. The applied electrospray voltage was 2.2 kV. No sheath gases or make up flows were applied, though the mass spectrometer's heated capillary was operated at 150° C. The sample was sprayed into a Model TSQ7000, (Finnigan, San Jose, Calif.).

The third quadrupole of the mass spectrometer was scanned over the mass to charge range of 475 to 1800 in 1.0 sec. If ions present in this mass range exceeded 80,000 counts, then the three most intense ions present in the spectra were subjected to collision induced dissociation. The collision cell was operated at ˜3 mtorr, while the applied collision voltage was adjusted for each precursor ion by multiplying each ion's mass to charge ratio by a factor of 26. The scanned range for the MS/MS scans were also mass to charge dependent, scanning up to a ratio twice that of the precursor ion's apparent mass to charge.

The mass spectral data was analyzed by SEQUEST (version 27PVM, Finnigan) on a supercomputer. The output files were then each viewed to verify the accuracy of the protein assignments. The MS analysis and identification of all spots in shown in Table I.

TABLE I.
Proteomic Analysis of RANKL-Induced Signal Transduction
Intermediates

| Protein Identified | Function |
|---|---|
| Tubulin alpha-1 chain-homo sapiens | Cytoskeletal rearrangement |
| Tubulin beta-3 chain | Cytoskeletal rearrangement |
| Alpha-2-HS-glycoprotein precursor | Regulatory |
| Actin Alpha | Cytoskeletal rearrangement |
| Tropomyosin, Cytoskeletal type | Cytoskeletal rearrangement |
| DNA mismatch repair protein MSH3 | Repair |
| Keratin Type II Cytoskeletal | Cytoskeletal rearrangement |
| Glyceraldehyde 3-phosphate | Cytoskeletal rearrangement |
| dehydrogenase | |
| Alfa-fructose bisphosphate aldolase | Cytoskeletal rearrangement |
| KPY1-pyruvate kinase M1 isozyme | Cytoskeletal rearrangement |
| Vimentin | Cytoskeletal rearrangement |
| Alpha enolase | Cytoskeletal rearrangement |
| Beta enolase | Cytoskeletal rearrangement |
| Macrophage capping protein | Cytoskeletal rearrangement |
| (gelsolin) | |
| RHO GDP-dissociation inhibitor 1 | Cytoskeletal rearrangement |
| 14-3-3 protein | Cytoskeletal rearrangement |
| Cofilin, non-muscle isoform | Cytoskeletal rearrangement |
| RAB-5C | Cytoskeletal rearrangement |
| Galectin-3 | Regulatory tyrosine phosphatase |
| Thioredoxin peroxidase I | Regulatory NF-κB |
| Proteasome beta chain | Regulatory 1kB |
| Endoplasmic reticulum protein ERP29 | Regulatory Stress Response |

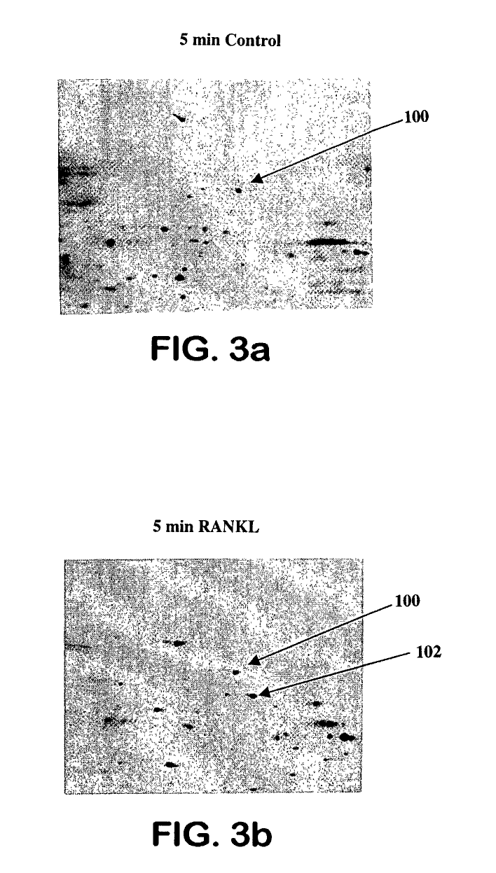

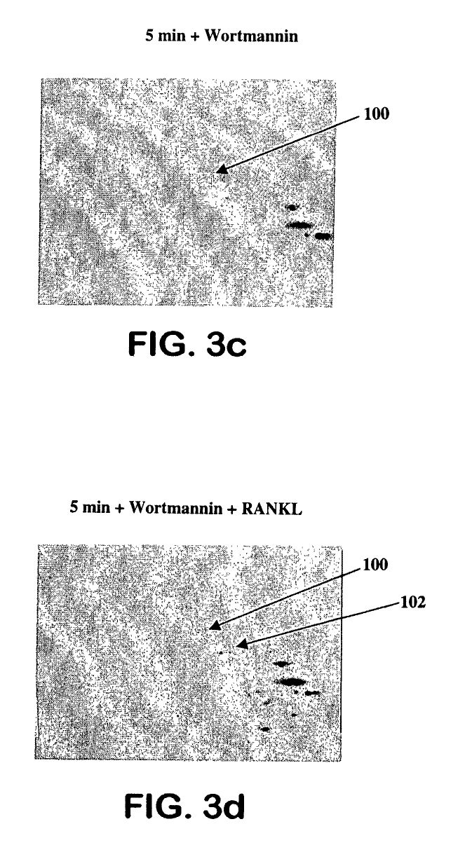

Western blot data showing phosphotyrosine activity in the presence or absence of wortmannin is shown in FIGS. 3a-3d. FIG. 3a shows an unstimulated (control) blot with α-enolase 100 identified as a landmark. Stimulation with RANKL causes capG 102 phosphorylation (FIG. 3b). As seen in FIGS. 3c and 3d, wortmannin has a pronounced effect on the phosphorylation pattern on the control unstimulated cells, as well as on the RANKL stimulated RAW 264 cells, respectively. The increase in capG phosphorylation in RANKL-stimulated cells and the decrease in capG phosphorylation in RANKL-stimulated cells the presence of wortmannin further indicates that capG is a critical enzyme in the RANKL pathway.

As shown in FIGS. 1, 2a-2d and 3a-3d, as well as in Table I, proteins which appeared to be differentially phosphorylated by RANKL treatment yet nonphosphorylated or reduced in phosphorylation by pretreatment with PP1 or wortmannin consisted primarily of cytoskeletal proteins, including capG. This suggested that src, itself a tyrosine kinase and PI3kinase, a phospholipid kinase, was upstream of one or more tyrosine kinases which were dependent upon the generation of phosphotidylinositol 3 phosphate (PtdIns-3-P), Phosphotidylinositol 3, 4 bis-phosphate (PtdIns-3,4-P₂) or Phosphotidylinositol 3, 4, 5 tris-phosphate (PtdIns-3,4,5-P₃) by Class I, II, and or III PI3kinases.

Wortmannin inhibition effectively blocks the ability of PI3kinase to phosphorylate the D-3 position of the inositol ring of PtdIns to PtdIns-3-P (PIP₂), PtdIns-4-P to PtdIns-3,4-P₂ (PIP₂), and PtdIns-4,5-P₂ (PIP₂) to PtdIns-3,4,5-P₃ (PIP₃) (Reinhold, S. L. et al. “Activation of human neutrophil phospholipase D by three separable mechanisms”. FASEB J. 4(2):208-214 (1990); Arcaro, A. et al. “Wortmannin is a potent phosphatidylinositol 3-kinase inhibitor: the role of phosphatidylinositol 3,4,5-trisphosphate in neutrophil responses.” Biochem J. 296 (Pt 2):297-301 (1993)). Additionally, capG was noted to be differentially phosphorylated and dependent upon the presence of PIP₃ based upon observations from wortmannin pretreatment.

A DNA sequence alignment of capG to gelsolin was conducted, as shown in FIG. 4. The DNA sequence information was obtained from NCBI for human and mouse capG and gelsolin was aligned with homologous regions indicated in the figure. As shown in FIG. 4, capG reflects sequence similarity to gelsolin.

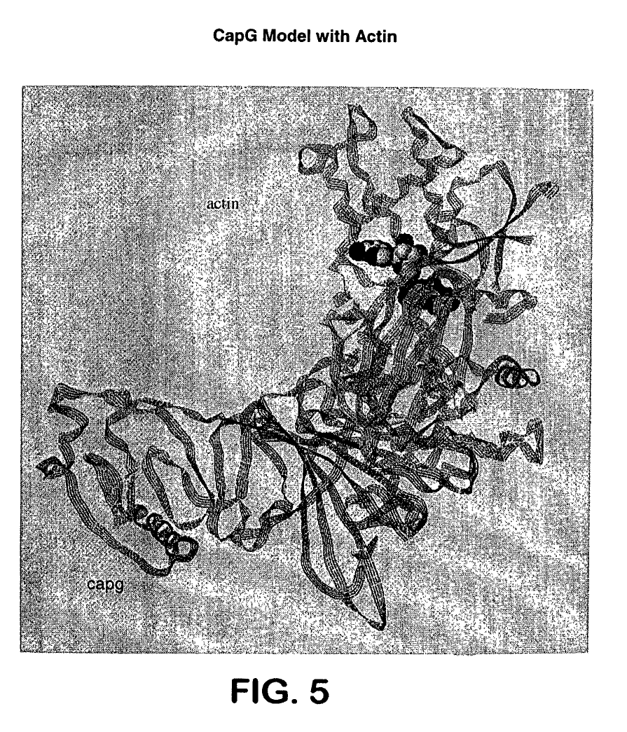

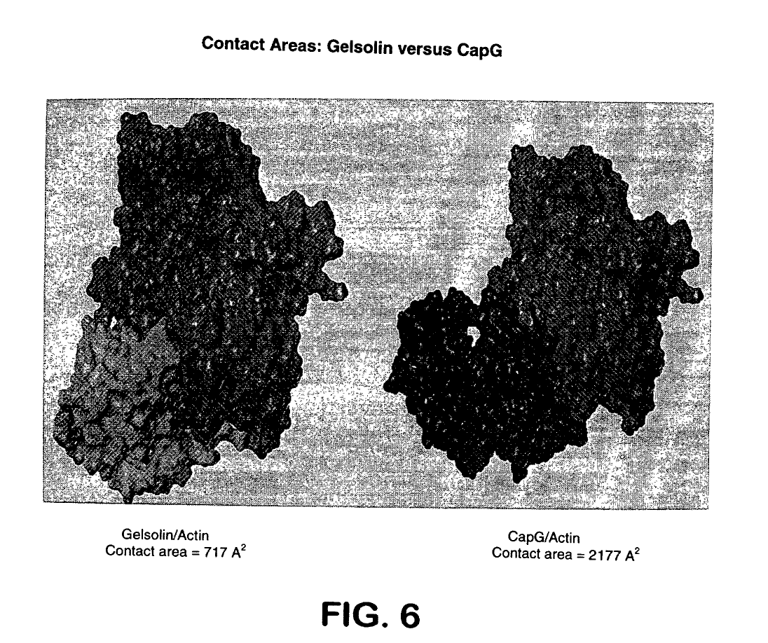

As shown in FIG. 5, a three dimensional alignment of capG was projected onto actin. The three dimensional structures for gelsolin and capG were then projected onto actin, as shown in FIG. 6. It is observed that the gelsolin actin contact area is caluculated as 717 square Angstroms, whereas the contact area of capG actin is calculated at 2177 square Angstroms. This model illustrates that the interaction between capG and actin is specific and that an extended contact area may be utilized for specific compound targeting.

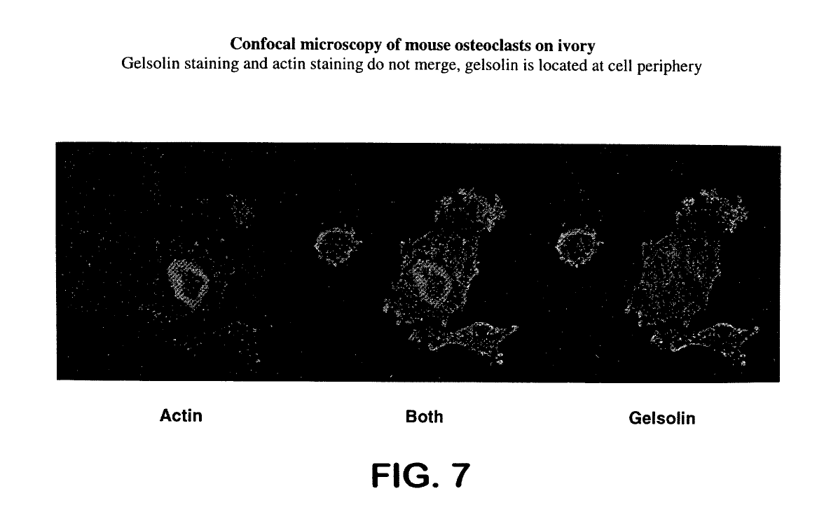

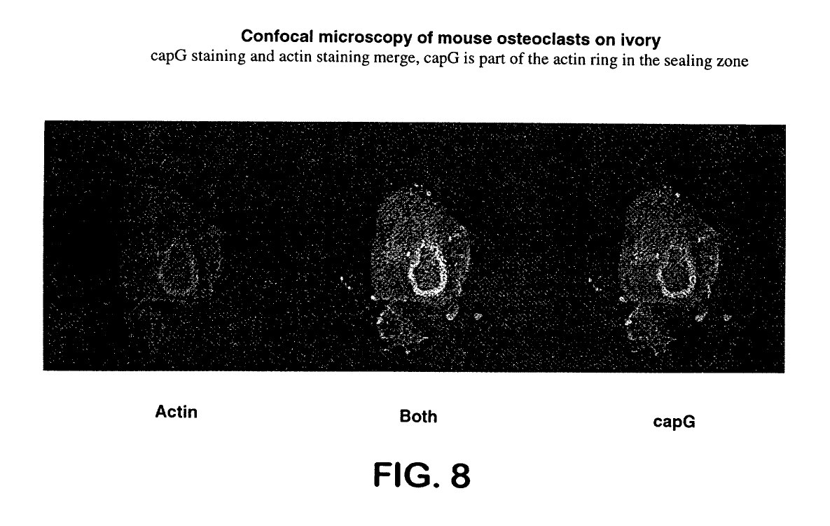

The inferred specificity of capG versus gelsolin is confirmed using confocal microscopy, as shown in FIGS. 7 and 8. As shown in FIG. 7, mouse activated osteoclasts were plated onto ivory, a bone resorption substrate. The cells were then immuno-stained for both actin and gelsolin. The image shows that gelsolin staining is confined to the podosomes and periphery of the cell membrane, whereas the actin staining is localized to the sealing zones, specific structures which the osteoclasts form on the bone matrix to initiate bone resorption. As shown in FIG. 8, the activated osteoclasts were immuno-stained for both capG and gelsolin. The image shows that both the capG and actin stains coincide with the sealing zone. Therefore, the capG protein is a specific component of the actin ring necessary to the formation of the sealing zone.

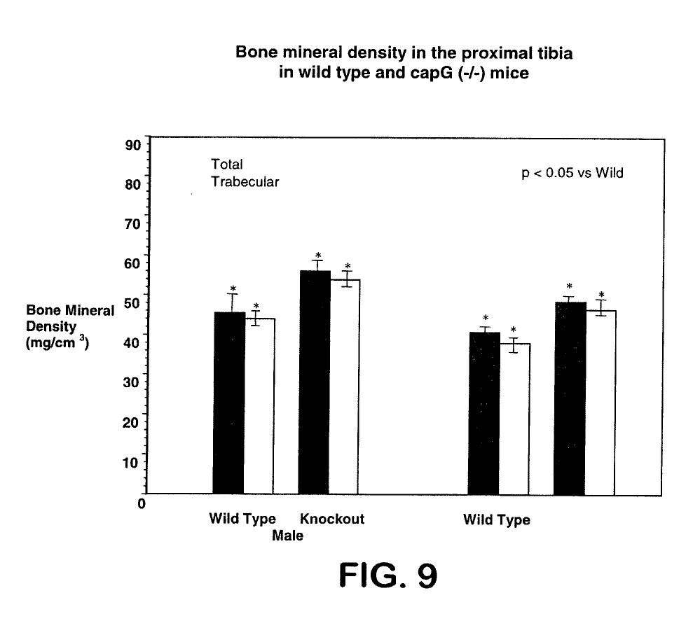

FIG. 9 shown bone mineral density analysis of tibia isolated from capG (−/−) and wild type mice. This analysis shows that the capG knockout mutation results in a state of osteopetrosis indicating abnormal osteoclast activation and functionality. This confirms the importance of capG in osteoclast activation and subsequent bone resorption. Therefore, capG is a useful marker and target protein for osteoporosis, and plays an important role in the treatment and prevention of osteoporosis and related disease states.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. An assay for identifying a compound that modulates the activity of capG, comprising:
(1) providing a cell expressing capG;
(2) contacting said cell expressing capG with a test compound; and
(3) determining whether said test compound interacts with capG.

2. The assay of claim 1, wherein said assay is a cell-based assay.

3. The assay of claim 1, wherein said assay is a cell-free assay.

4. The assay of claim 3, wherein said cell-free assay is a ligand-binding assay.

5. The assay of claim 1, wherein said test compound modulates the activity of capG.

6. The assay of claim 1, wherein said test compound is a capG antagonist.

7. The assay of claim 1, wherein said test compound is a capG agonist.

8. The assay of claim 1, wherein said test compound binds to capG.

9. The assay of claim 1, wherein said test compound inhibits the activity of capG.

10. The assay of claim 1, wherein said assay is for identifying compounds which will be useful for the treatment of osteoporosis.

11. A method for the treatment of osteoporosis, comprising administering to a patient in need thereof a therapeutically effective amount of a compound which was identified by the assay of claim 1.

12. A method for the treatment of osteoporosis, comprising:
(1) identifying a patient suffering from osteoporosis; and
(2) administering to said patient a therapeutically effective amount of a modulator of capG.

13. The method of claim 12, wherein said patient is identified as suffering from osteoporosis by measuring the expression level of capG in said patient.

14. The method of claim 12, wherein said modulator is a capG antagonist.

15. A method for the prevention of osteoporosis, comprising:
(1) identifying a patient at risk for osteoporosis; and
(2) administering to said patient a therapeutically effective amount of a modulator of capG.

16. The method of claim 15, wherein said patient is identified as being at risk for osteoporosis by measuring the expression level of capG in said patient.

17. The method of claim 15, wherein said modulator is a capG antagonist.

18. A method for the prevention of osteoporosis, comprising:
(1) identifying a patient at risk for osteoporosis; and
(2) determining if said patient has increased expression of capG, wherein said increased expression is measured relative to the expression of capG in a patient not at risk for osteoporosis.

19. A method of decreasing the differentiation of osteoclast precursor cells into osteoclast cells, comprising contacting said osteoclast precursor cells with a capG modulator.

20. The method of claim 19, wherein said modulator is a capG antagonist.

21. A compound capable of modulating the activity of capG.

22. A compound of claim 21, wherein said compound is identified by:
(1) providing a cell expressing capG;
(2) contacting said cell expressing capG with said compound; and
(3) determining whether said compound modulates the activity of capG.

23. The compound of claim 21, wherein said compound is a capG antagonist.

24. The compound of claim 21, wherein said compound is a capG agonist.

25. The compound of claim 21, wherein said compound binds to capG.
